(19) United States
(12) Patent Application Publication
(10) Pub. No.: US 2016/0106106 A1
(43) Pub. Date: Apr. 21, 2016

(54) DRUG-RESISTANT MICROBE AND VARIANT MICROBE DISINFECTANT CONTAINING CHLOROUS ACID AQUEOUS SOLUTION

(71) Applicant: HONBU SANKEI CO., LTD., Osaka (JP)

(72) Inventor: Hisataka GODA, Osaka (JP)

(21) Appl. No.: 14/892,324

(22) PCT Filed: May 15, 2014
(86) PCT No.: PCT/IB2014/061451

§ 371 Date: Nov. 19, 2015

(30) Foreign Application Priority Data
May 20, 2013 (JP) 2013-106208
May 20, 2013 (JP) 2013-106214
Nov. 11, 2013 (JP) 2013-232955

(51) Int. Cl. A01N 59/00

(52) U.S. Cl. CPC A01N 59/00

(57) ABSTRACT

The present invention provides microbe disinfectants, providing: Drug-resistant microbe disinfectants comprising a chlorous acid aqueous solution for inactivating microbes selected from methicillin-resistant Staphylococcus aureus, multidrug-resistant Pseudomonas aeruginosa, and vancomycin-resistant Enterococcus; and microbe disinfectants, which are made with acidity when applied to gram-negative microbes and with alkalinity when applied to gram-positive microbes. The microbes comprise at least one species of microbes selected from the group consisting of E. coli, Staphylococcus aureus, microbes of genus Bacillus, microbes of genus Paenibacillus, Pseudomonas aeruginosa, Enterococcus, Salmonella enterica, and periodontal disease microbes. The present invention is usable as a microbe disinfectant that is safe to human body and easy to handle as a microbe disinfectant for pretreatment in food processing and produces chlorous acid that generates little chlorine dioxide. The microbe disinfectant comprising a chlorous acid aqueous solution of the present invention can be utilized as a sterilizing agent, food additive, antiseptic, quasi-drug, medicine, etc.

The present invention relates to a drug-resistant microbe disinfectant comprising a chlorous acid aqueous solution.

The present invention relates to a variant microbe disinfectant comprising a chlorous acid aqueous solution.

### BACKGROUND ART

The issues related to drug-resistant microbes are old and new problems. Although antibiotics are excellent drugs, an issue associated therewith is that target microbes gradually acquire resistance. Historically, beginning with Staphylococcus aureus acquiring resistance to penicillin in the 1950s (penicillin-resistant Staphylococcus aureus), acquisition of resistance to methicillin was discovered in the 1970s (methicillin-resistant Staphylococcus aureus). Thereafter, resistance to vancomycin was found in the 1990s (vancomycin-resistant Enterococcus (VRE), vancomycin intermediate-resistant Staphylococcus aureus (VISA), 1997). Further, vancomycin-resistant Staphylococcus aureus was reported in 2002, and it became a world-wide issue. In this manner, antibiotics tend to become a cat-and-mouse game. Thus, drug resistance is an issue for antibiotics.

Further, a chlorous acid aqueous solution has been registered recently as a food additive. Since a chlorous acid aqueous solution has an effect as it is, a chlorous acid aqueous solution, in many cases, is used directly as the method of use thereof.

The inventor has discovered a method of manufacturing a chlorous acid aqueous solution. A sterilizing effect against E. coli was verified and a patent application therefor was filed (Patent Literature 1).

### CITATION LIST

### Patent Literature

### [PTL 1]

International Publication No. WO 2008-026607

### SUMMARY OF INVENTION

### Solution to Problem

The present invention provides a microbe disinfectant capable of unexpectedly and significantly disinfecting drug-resistant microbes extensively. The present invention also provides the following.

(1) A drug-resistant microbe disinfectant comprising a chlorous acid aqueous solution.
(2) The drug-resistant microbe disinfectant of (1), wherein the drug-resistant microbe disinfectant inactivates microbes selected from methicillin-resistant Staphylococcus aureus, multidrug-resistant Pseudomonas aeruginosa, and vancomycin-resistant Enterococcus.
(3) The drug-resistant microbe disinfectant of (1) or (2), wherein the drug-resistant microbe disinfectant is present at at least 100 ppm.
(4) The drug-resistant microbe disinfectant according to any one of (1) to (3), wherein the drug-resistant microbe disinfectant is present at at least 200 ppm.
(5) The drug-resistant microbe disinfectant according to any one of (1) to (4), wherein the drug-resistant microbe disinfectant is present at at least 500 ppm.
(6) The drug-resistant microbe disinfectant according to any one of (1) to (5), wherein the drug-resistant microbe disinfectant inactivates methicillin-resistant Staphylococcus aureus and pH is 6.5 or higher.
(7) The drug-resistant microbe disinfectant according to any one of (1) to (6), wherein the drug-resistant microbe disinfectant inactivates microbes selected from multidrug-resistant Pseudomonas aeruginosa and vancomycin-resistant Enterococcus and pH is 6.5 or lower.
(8) The drug-resistant microbe disinfectant according to any one of (1) to (7), wherein pH is about 6.5.
(9) The drug-resistant microbe disinfectant according to any one of (1) to (8), wherein the drug-resistant microbe disinfectant is a disinfectant for drug-resistant microbes in urine.

Further, when the present invention is used as a microbe disinfectant, a microbe disinfecting effect was unexpectedly found to be enhanced by making the disinfectant acidic when applied to gram-negative microbes and approximately neutral when applied to gram-positive microbes. This is thus provided as the present invention. Further, it was found that the present invention additionally has an effect on various microbes to which an effect has not been shown conventionally. Thus, the present invention provides the application thereof. The present invention also provides the following.

(1) A microbe disinfectant comprising a chlorous acid aqueous solution, wherein the microbe disinfectant is made with acidity when applied to gram-negative microbes or with neutrality when applied to gram-positive microbes.
(2) The microbe disinfectant of (1), wherein the acidity is pH of 6.5 or lower and the neutrality is pH of 6.5 or higher.
(3) The microbe disinfectant of (1) or (2), wherein the microbe disinfectant is provided as a kit comprising a chlorous acid aqueous solution and an agent imparting acidity and/or neutrality.
(4) The microbe disinfectant according to anyone of (1) to (3), wherein the microbes comprise pathogenic microbes.
(5) The microbe disinfectant according to anyone of (1) to (4), wherein the microbes comprises at least one species of microbes selected from the group consisting of E. coli, Staphylococcus aureus, microbes of genus Bacillus, microbes of genus Paenibacillus, Pseudomonas aeruginosa, Enterococcus, Salmonella enterica, Campylobacter, and periodontal disease microbes.
(6) A periodontal disease microbe disinfectant comprising a chlorous acid aqueous solution.
(7) The disinfectant according to any one of (1) to (6), wherein pH is about 6.5.
(8) A microbe disinfecting kit, comprising a pH adjusting agent and a disinfectant according to any one of (1) to (7).
(9) A microbe disinfectant comprising a chlorous acid aqueous solution, wherein the disinfectant is made to contact target microbes at a concentration of at least 25 ppm upon contact.
(10) The microbe disinfectant of (9), wherein the concentration is at least 50 ppm.

In the present invention, one or more features described above are intended to provide combinations that were explicitly described as well as combinations thereof. The additional embodiments and advantages of the present invention are recognized by those skilled in the art if the following Detailed Description is read and understood as needed.

### Advantageous Effects of Invention

According to the present invention, a microbe disinfectant with the ability to disinfect highly drug-resistant microbes is provided. Further, the present invention provides a microbe disinfectant with suppressed chlorine dioxide generation, which can be reliably used and is safe in a human body. Such a microbe disinfectant can be utilized as a microbe disinfectant that can be widely used in clinical practice or the like.

The issues inherent in sodium hypochlorite and alcohol that exhibit extensive sterilizing power have been resolved. That is, although there was an issue of sodium hypochlorite not being safe to a human body, this has been resolved. Further, when the alcohol concentration is 60% or higher, alcohol is hazardous and difficult to handle. In addition, when the concentration is less than 60%, it was difficult to obtain a microbe disinfecting effect. However, a microbe disinfectant that is equally or safer and more powerful in comparison thereto is provided.

A chlorous acid aqueous solution has an excellent microbe disinfecting effect against numerous drug-resistant microbes, especially against multidrug-resistant microbes.

A chlorous acid aqueous solution has an excellent microbe disinfecting effect against periodontal disease microbes, Pseudomonas aeruginosa in urine, and multidrug-resistant microbes. The microbe disinfecting effect of a chlorous acid aqueous solution was found to having a tendency to be strong on the acidic side (approximately pH of 6.5 or lower) against gram-negative microbes and strong in the neutral range (approximately pH 6.5 or higher) against gram-positive microbes. An advantageous method of use as a microbe disinfecting agent is provided based on this discovery.

A chlorous acid aqueous solution has a potential as a growth suppressing substance against Pseudomonas aeruginosa in urine.

### BRIEF DESCRIPTION OF DRAWINGS

FIG. 1 shows a scheme for examining a micro disinfecting effect of a chlorous acid aqueous solution on multidrug-resistant microbes.

FIG. 2 shows a microbe disinfecting effect of a chlorous acid aqueous solution with regard to Methicillin-resistant Staphylococcus aureus (MRSA) COL. Top left is data for a chlorous acid aqueous solution expressed in concentration (expressed in ppm). Top right, bottom left, and bottom right show data for a chlorous acid aqueous solution (left) and sodium chlorite (right) at 100 ppm, 200 ppm, and 500 ppm, respectively. From the left, pH of 8.5, 7.5, 6.5, 5.5, and 4.5 is shown.

FIG. 3 shows a microbe disinfecting effect of a chlorous acid aqueous solution with regard to Multidrug-resistant Pseudomonas aeruginosa (MDRP) TUH. Top left is data for a chlorous acid aqueous solution expressed in concentration (expressed in ppm). Top right, bottom left, and bottom right show data for a chlorous acid aqueous solution (left) and sodium chlorite (right) at 100 ppm, 200 ppm, and 500 ppm, respectively. From the left, pH of 8.5, 7.5, 6.5, 5.5, and 4.5 is shown.

FIG. 4 shows a microbe disinfecting effect of a chlorous acid aqueous solution with regard to Vancomycin-resistant Enterococcus faecalis BM1447. Top left is data for a chlorous acid aqueous solution expressed in concentration (expressed in ppm). Top right, bottom left, and bottom right show data for a chlorous acid aqueous solution (left) and sodium chlorite (right) at 100 ppm, 200 ppm, and 500 ppm, respectively. From the left, pH of 8.5, 7.5, 6.5, 5.5, and 4.5 is shown.

FIG. 5 shows the results of examining the growth suppressing effect of a chlorous acid aqueous solution on contaminating microbes in urine (MDRP). The figure shows: only microbial solution; chlorous acid aqueous solution (10 ppm, 50 ppm, 100 ppm), sodium chlorite (10 ppm, 50 ppm, 100 ppm); and sodium hypochlorite (10 ppm, 50 ppm, 100 ppm).

FIG. 6 shows the results of examining a round test that is different from those of FIG. 5 with respect to the growth suppressing effect of a chlorous acid aqueous solution on contaminating microbes in urine (MDRP, MRSA). The figure shows: only microbial solution; chlorous acid aqueous solution (10 ppm, 50 ppm, 100 ppm), sodium chlorite (10 ppm, 50 ppm, 100 ppm); and sodium hypochlorite (10 ppm, 50 ppm, 100 ppm).

FIG. 7 is a graph for absorbance and wavelength from a component analysis confirmation test (Table 2, Confirmation Test (2)) for a chlorous acid aqueous solution.

FIG. 8 is a graph for absorbance and wavelength from a confirmation test (Table 4, Confirmation Test (2)) for a chlorous acid aqueous solution.

FIG. 9 shows an experimental example for periodontal disease microbes (Fusobacterium nucleatum F-1) with a chlorous acid aqueous solution. The left side shows the protocol and the right side shows the survival rate in a buffer in a pentagonal shape (control only having buffer).

FIG. 10 shows an experimental example for periodontal disease microbes (Fusobacterium nucleatum F-1) with a chlorous acid aqueous solution. Top left shows chlorous acid aqueous solution, top right shows sodium hypochlorite, bottom left shows high-grade chlorinated lime, and bottom right shows sodium chlorite.

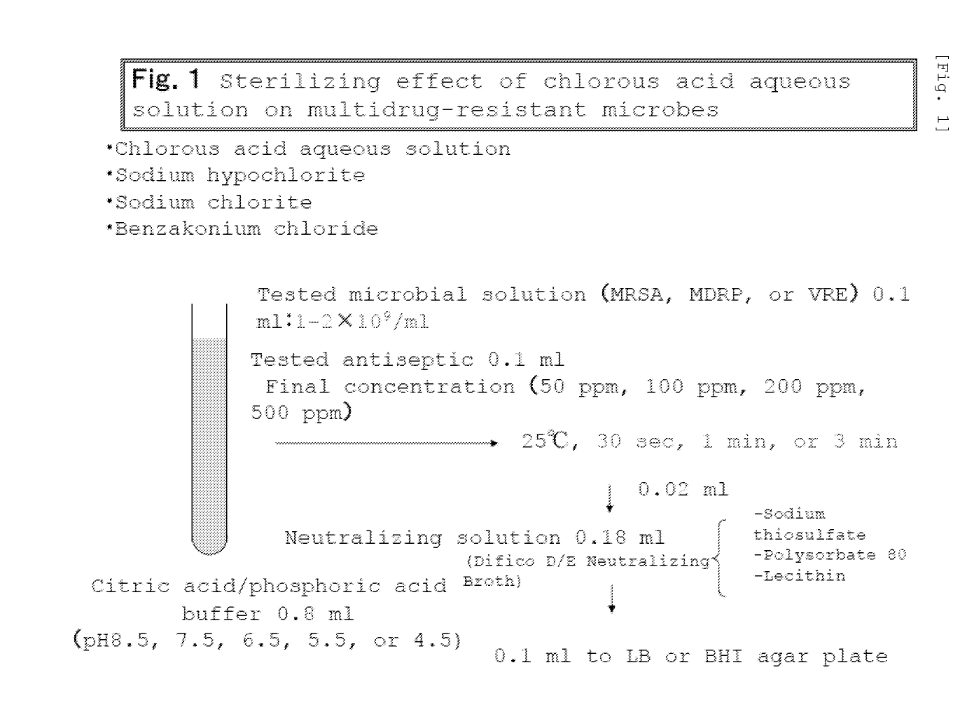

### DESCRIPTION OF EMBODIMENTS

The present invention is described below. Throughout the entire specification, a singular expression should be understood as encompassing the concept thereof in a plural form unless specifically noted otherwise. Thus, singular articles (e.g., “a”, “an”, the and the like in case of English) should be understood as encompassing the concept thereof in a plural form unless specifically noted otherwise. Further, the terms used herein should be understood as being used in the meaning that is commonly used in the art, unless specifically noted otherwise. Thus, unless defined otherwise, all terminologies and scientific technical terms that are used herein have the same meaning as the terms commonly understood by those skilled in the art to which the present invention belongs. In case of a contradiction, the present specification (including the definitions) takes precedence.

Herein, “drug resistance” refers to a phenomenon of having resistance to a drug, such as antibiotics, having some type of an effect on a microbe itself and thereby such drugs becoming ineffective or less effective thereon.

Herein, a “drug-resistant microbe” refers to a microbe that has acquired drug-resistance. Such a drug-resistant microbe includes, but not limited to, methicillin-resistant Staphylococcus aureus (MRSA), multidrug-resistant Pseudomonas aeruginosa (MDRP), vancomycin-resistant Enterococcus (VRE), and Clostridium difficile (CD: sporulation, toxicogenic). Although it is not desired to be constrained by theory, a drug-resistant gene generally has acquired a gene imparting drug-resistance. The present invention is considered to have a microbe disinfecting effect by destroying such a gene or gene product. Thus, in the present invention, an effect on specific multidrug resistant microbes, which are microbes that can resist multiple drugs, is demonstrated in the Examples. Those skilled in the art understand that the present invention is extrapolated to generally have an effect on simpler drug-resistant microbes.

Herein, a “multidrug-resistant microbe” refers to a microbe that has acquired drug-resistance to multiple drugs (especially antibiotics).

Herein, “antimicrobial (action)” refers to suppression of growth against microorganisms such as mold, microbes, or viruses that are pathogenic or harmful, especially against microbes. A substance having antimicrobial action is referred to as an antimicrobial agent.

Herein, “sterilizing (action)” refers to killing of microorganisms such as mold, microbes, or viruses that are pathogenic or harmful, especially of microbes. A substance having sterilizing action is referred to as a sterilizing agent.

Antimicrobial action and sterilizing action on microbes are together referred to as microbe disinfecting (action). Thus, a substance having antimicrobial action and sterilizing action on microbes is generally referred herein as a “microbe disinfecting agent”. Thus, substances against drug-resistant microbes are called, for example, “drug-resistant microbe disinfectant”. Microbe disinfectants encompass drug-resistant microbe disinfectants.

Multidrug-resistant Pseudomonas aeruginosa is one species of “Pseudomonas aeruginosa”. Pseudomonas aeruginosa is extensively found in the natural world. The nutritional demand of Pseudomonas aeruginosa is low and Pseudomonas aeruginosa can grow in water that barely contains any nutrients. Pseudomonas aeruginosa is characterized by its production of green pigments (pyocyanin) and formation of a biofilm. Multidrug-resistant Pseudomonas aeruginosa (MDRP) refers to Pseudomonas aeruginosa that exhibits resistance to all of carbapenem, fluoroquinolone, and aminoglycoside lines, which are three lines of antimicrobial agents that have conventionally exhibited high antimicrobial activity against Pseudomonas aeruginosa.

The determination baseline for MDRP is as shown in the following Table.

TABLE 1

| Antimicrobial Agent | MIC (ug/ml) |
|---|---|
| Imipenem | ≧16 |
| Amikacin | ≧32 |
| Ciprofloxacin | ≧4 |

Methicillin-resistant Staphylococcus aureus (MRSA) is a species of Staphylococcus aureus and refers to Staphylococcus aureus that has acquired drug resistance to the antibiotic methicillin. However, methicillin-resistant Staphylococcus aureus (MRSA) is in fact a multidrug-resistant microbe that exhibits resistance to many antibiotics. Typical therapeutic agents are vancomycin, teicoplanin, and arbekacin. However, strains resistant to vancomycin have appeared. MRSA succeeded in acquiring resistance to methicillin by employing a strategy that is different from conventional penicillin-resistant microbes. Unlike conventional staphylococcus, MRSA avoids the effect of a β-Lactam agent by making peptidoglycan synthase (PBP2′) to which a β-Lactam agent cannot bind. The protein PBP2′ is encoded by a gene called mecA. Thus, MRSA can be identified by the presence of the protein PEBP2′ or the presence of the gene mecA. A method of determination includes a determination based on results of drug sensitivity tests and determination by detection of an MRSA specific gene. Drug sensitivity tests determine Staphylococcus by an identification testing method commonly practiced at each medical facility and determine as MRSA when an MIC value of oxacillin of 4≧μg/ml is exhibited after culturing for 24 hours at 35° C. in the presence of 2% NaCl in accordance with the standard method of NCCLS (National Committee for Clinical Laboratory Standards). Further, determination of MRSA is made when a diameter of a zone of inhibition of oxacillin is ≦10 mm under similar culturing conditions when using NCCLS-specified disk diffusion method. Alternatively, in determination by detecting an MRSA specific gene, it is possible to utilize of method of simultaneously detecting a mecA gene (gene of PBP2′ associated with methicillin resistance) and a Staphylococcus aureus-specific gene (spa gene=staphylococcal protein A gene) by PCR or the like.

Staphylococcus aureus found in urine is especially called Staphylococcus aureus in urine.

Vancomycin-resistant Enterococcus (VRE) is a species of Enterococcus that has acquired drug-resistance to vancomycin. Enterococcus is a type of resident flora present in the intestine of a human being or an animal. In a healthy normal body, Enterococcus does not become a factor in inducing an infectious disease. However, in a state of decreased immunity due to some type of sickness, Enterococcus can induce endocarditis, septicemia, urinary tract infection or the like. Resistance is exhibited against drugs such as ampicillin, vancomycin, new quinolone, carbapenem and the like that are effective against normal Enterococcus (especially faecalis). Enterococcus having VanA and VanB genes has become issues. Thus, it is possible to identify VRE by a gene detection method similar to that for MRSA.

Periodontal diseases are induced by various microbes. Herein, microbes that cause a periodontal disease are together called periodontal disease microbes. For example, periodontal disease microbes include Aggregatibacter actinomycetemcomitans, Prophyromonas gingivaris, Tannerella forsythensis, Treponema denticola, Prevotella intermedia, Fusobacterium nucleatum, Campylobacter rectus, Eikenella corrodens, Actinomyces genus and the like. Typically, tests can use Fusobacterium nucleatum F-1. Fusobacterium nucleatum F-1 is an obligate anaerobic gram-negative bacillus, Vincent's angina (acute tonsillitis from combined infections of Fusobacterium and Borrelia vincentii; referred to as ulceromembranous tonsillitis, necrotizing ulcerative tonsillitis). In the present invention, a chlorous acid aqueous solution exhibits an excellent microbe disinfecting effect on periodontal disease microbes. The effect thereof is equivalent to or greater than that of sodium hypochlorite. It has been demonstrated to decrease the number of surviving microbes to 10⁻⁵ or less in 30 minutes.

The microbes targeted by the chlorous acid aqueous solution of the present invention may be E. coli (Escherichia coli), Staphylococcus aureus (Staphylococcus aureus and the like), microbes of the genus Bacillus (Bacillus sp.), microbes of the genus Paenibacillus (Paenibacillus sp.), Pseudomonas aeruginosa (Pseudomonas aeruginosa and the like), Enterococcus (Enterococcus faecalis and the like), Salmonella (Salmonella sp.), Campylobacter (Campylobacter sp.), periodontal disease microbes (Fusobacterium nucleatum and the like) or the like.

Herein, a “pathogenic microbe” refers to any microbe that can cause a disease. When a pathogenic microbe is targeted, since the microbe disinfectant of the present invention can target pathogenic microbes, the microbe disinfectant of the present invention can be used in pharmaceutical applications.

Herein, “acidic (region)”, when used with regard to the microbe disinfectant of the present invention, refers to a chlorous acid aqueous solution having pH that is more acidic than pH of 6.5, which is considered a neutral region. For example, such pH includes, but is not limited to, pH of 6.5 or lower, pH of 6.4 or lower, pH of 6.3 or lower, pH of 6.2 or lower, pH of 6.1 or lower, pH of 6.0 or lower, pH of 5.9 or lower, pH of 5.8 or lower, pH of 5.7 or lower, pH of 5.6 or lower, pH of 5.5 or lower, pH of 5.4 or lower, pH of 5.3 or lower, pH of 5.2 or lower, pH of 5.1 or lower, pH of 5.0 or lower, pH of 4.9 or lower, pH of 4.8 or lower, pH of 4.7 or lower, pH of 4.6 or lower, pH of 4.5 or lower, and the like.

Herein, “neutral (region)”, when used with regard to the microbe disinfectant of the present invention, refers to a chlorous acid aqueous solution having pH at about 6.5 or more in the range towards the alkaline side. For example, such pH includes, but not limited to, pH of 6.5 or higher, pH of 6.6 or higher, pH of 6.7 or higher, pH of 6.8 or higher, and pH of 6.9 or higher, and the upper limit includes pH of 8.5 or less, pH of 8.4 or less, pH of 8.3 or less, pH of 8.2 or less, pH of 8.1 or less, pH of 8.0 or less, pH of 7.9 or less, pH of 7.8 or less, pH of 7.7 or less, pH of 7.6 or less, pH of 7.5 or less, pH of 7.4 or less, pH of 7.3 or less, pH of 7.2 or less, pH of 7.1 or less, pH of 7.0 or less, pH of less than 7.0 and the like. Since the present invention uses a chlorous acid aqueous solution, pH of less than 7.0 is preferable, although but not limited thereto, to distinguish from sodium chlorite.

For example, acidity and neutrality can be adjusted by using a buffering system such as citric acid buffer, phosphoric acid buffer, or citric acid/phosphoric acid buffer. A buffer system can be made by adding a citric acid metal salt (e.g., sodium citrate) to citric acid in a citric acid buffer, or a phosphoric acid metal salt (e.g, sodium phosphate) to phosphoric acid in a phosphoric acid buffer. In addition, a citric acid/phosphoric acid buffer can be adjusted by appropriately combining the two.

Herein, an “agent imparting acidity and/or neutrality” can be any agent that can adjust the pH of a chlorous acid aqueous solution. An agent that imparts acidity and an agent that imparts neutrality may be separately comprised, but may be an agent that can adjust pH to a desirable value by using a buffering system. Thus, an agent imparting acidity and/or neutrality can include, but not limited to, an agent for manufacturing a buffering system, e.g., a combination of citric acid and citric acid metal salt, a combination of phosphoric acid and a phosphoric acid buffer, a combination thereof or the like.

Herein, an “agent imparting acidity” is an agent that can lower the pH of a chlorous acid aqueous solution, including but not limited to any inorganic acid and organic acid.

Herein, an “agent imparting neutrality” is an agent that can raise the pH of a chlorous acid aqueous solution, including but not limited to any salt of inorganic acid or organic acid and any inorganic base or organic base.

In order to achieve “making (a disinfectant) with acidity when applied to gram-negative microbes and with neutrality when applied to gram-positive microbes” in the present invention, a chlorous acid aqueous solution may be initially prepared to be acidic or neutral in accordance with the target. Alternatively, an agent imparting acidity may be appropriately added to make it acidic or an agent imparting neutrality may be added to make it neutral at the time of use.

Thus, the microbe disinfectant of the present invention can be provided as a kit comprising a chlorous acid aqueous solution and a pH adjusting agent. A pH adjusting agent can comprise an agent imparting acidity and/or an agent imparting neutrality.

Alternatively, the microbe disinfectant of the present invention comprises a chlorous acid aqueous solution with a pH of about 6.5. The microbe disinfectant of the present invention is preferred for use as an all-purpose agent because a microbe disinfectant that can disinfect not only gram-negative microbes, but gram-positive microbes can be provided due to the pH thereof being about 6.5. Herein, pH of “about” 6.5 refers to a range spanning 0.5 in both directions, including but not limited to pH of 6.0 to 7.0, 6.1 to 6.9, 6.2 to 6.8, 6.3 to 6.7, and 6.4 to 6.6. In addition, it is understood that any combination of these upper and lower limits may be used.

Herein, a “kit” refers to a unit that is generally divided into two or more sections to provide portions to be provided (e.g., chlorous acid aqueous solution (microbe disinfectant), pH adjusting agent (agent imparting acidity and/or agent imparting neutrality), manual, and the like). When it is intended to provide a composition which should not be provided in a mixed state and is preferably used by mixing immediately prior to use, or when it is intended to provide a composition for which an appropriate adjustment of pH is preferably performed immediately prior to use, such a kit form is preferred. It is preferable and advantageous for such a kit to comprise, for example, an instruction or manual describing a method of use, method of adjustment and the like.

Herein, an “instruction” describes an explanation regarding a method of using the present invention for a user. The instruction has descriptions instructing a method of preparing the present invention, usage of microbe disinfectant and the like. The instruction is made according to a format stipulated by the regulatory agency of the county in which the present invention is carried out (e.g., Ministry of Health, Labor and Welfare in Japan, Food and Drug Administration (FDA) in the United States, etc.). In addition, it may be explicitly described that an approval was received from said regulatory agency. An instruction is a so-called attached document (package insert), which is generally provided in, but not limited to, a paper medium. For example, such a document can also be provided in a form of an electronic medium (e.g., website provided through the internet, email, or SNS).

(Chlorous Acid Aqueous Solution and Manufacturing Example Thereof)

The chlorous acid aqueous solution used in the present invention has a feature that was discovered by the inventors. A chlorous acid aqueous solution manufactured by any method, such as known manufacturing methods described in Patent Literature 1, can be used. It is possible to mix and use an agent with, for example, 61.40% chlorous acid aqueous solution, 1.00% potassium dihydrogen phosphate, 0.10% potassium hydroxide, and 37.50% purified water, as a typical constitution (sold under the name “AUTOLOC Super” by the Applicant; 72% chlorous acid aqueous solution corresponds to chlorous acid at 30000 ppm), but the constitution is not limited thereto. The chlorous acid aqueous solution may be 0.25%-75%, potassium dihydrogen phosphate may be 0.70%-17.42%, and potassium hydroxide may be 0.10%-5.60%. It is possible to use sodium dihydrogen phosphate instead of potassium dihydrogen phosphate, or sodium hydroxide instead of potassium hydroxide. This agent can reduce the decrease of chlorous acid due to contact with an organic matter under acidic conditions. However, the sterilizing effect is retained. In addition, very little chlorine gas is generated. Further, the agent also has a feature of inhibiting amplification of odor from mixing chlorine with an organic matter.

In one embodiment, the chlorous acid aqueous solution of the present invention can be produced by adding and reacting sulfuric acid or an aqueous solution thereof to a sodium chlorate aqueous solution in an amount and concentration at which the pH value of the sodium chlorate aqueous solution can be maintained at 3.4 or lower to generate chloric acid, and subsequently adding hydrogen peroxide in an amount equivalent to or greater than the amount required for a reduction reaction of the chloric acid.

Further, in another embodiment, the chlorous acid aqueous solution of the present invention can be produced from adding one compound from inorganic acids or inorganic acid salts, two or more types of compounds therefrom, or a combination thereof to an aqueous solution, in which chlorous acid is produced by adding and reacting sulfuric acid or an aqueous solution thereof to a sodium chlorate aqueous solution in an amount and concentration at which the pH value of the sodium chlorate aqueous solution can be maintained at 3.4 or lower to generate chloric acid, and subsequently adding hydrogen peroxide in an amount equivalent to or greater than the amount required for a reduction reaction of the chloric acid, and adjusting the pH value within the range from 3.2 to 8.5.

Furthermore, in another embodiment, the chlorous acid aqueous solution of the present invention can be produced from adding one compound from inorganic acids or inorganic acid salts or organic acids or organic acid salts, two or more types of compounds therefrom, or a combination thereof to an aqueous solution, in which chlorous acid is produced by adding and reacting sulfuric acid or an aqueous solution thereof to a sodium chlorate aqueous solution in an amount and concentration at which the pH value of the sodium chlorate aqueous solution can be maintained at 3.4 or lower to generate chloric acid, and subsequently adding hydrogen peroxide in an amount equivalent to or greater than the amount required for a reduction reaction of the chloric acid, to adjust the pH value within the range from 3.2 to 8.5.

Further still, in another embodiment, the chlorous acid aqueous solution of the present invention can be produced from adding one compound from inorganic acids or inorganic acid salts or organic acids or organic salts, two or more types of compounds therefrom, or a combination thereof after adding one compound from inorganic acids or inorganic acid salts, two or more types of compounds therefrom or a combination thereof to an aqueous solution, in which chlorous acid is produced by adding and reacting sulfuric acid or an aqueous solution thereof to a sodium chlorate aqueous solution in an amount and concentration at which the pH value of the sodium chlorate aqueous solution can be maintained at 3.4 or lower to generate chloric acid, and subsequently adding hydrogen peroxide in an amount equivalent to or greater than the amount required for a reduction reaction of the chloric acid, and adjusting the pH value within the range from 3.2 to 8.5.

Further, in another embodiment, carbonic acid, phosphoric acid, boric acid, or sulfuric acid can be used as the inorganic acid in the above-described method.

Further still, in another embodiment, carbonate, hydroxy salt, phosphate or borate can be used as the inorganic acid salt.

Further, in another embodiment, sodium carbonate, potassium carbonate, sodium bicarbonate or potassium bicarbonate can be used as the carbonate.

Furthermore, in another embodiment, sodium hydroxide, potassium hydroxide, calcium hydroxide, or barium hydroxide can be used as the hydroxy salt.

Further still, in another embodiment, disodium hydrogen phosphate, sodium dihydrogen phosphate, trisodium phosphate, tripotassium phosphate, dipotassium hydrogen phosphate, or potassium dihydrogen phosphate can be used as the phosphate.

Further, in another embodiment, sodium borate or potassium borate can be used as the borate.

Furthermore, in another embodiment, succinic acid, citric acid, malic acid, acetic acid, or lactic acid can be used as the organic acid.

Further still, in another embodiment, sodium succinate, potassium succinate, sodium citrate, potassium citrate, sodium malate, potassium malate, sodium acetate, potassium acetate, sodium lactate, potassium lactate, or calcium lactate can be used as the organic acid salt.

In a method of manufacturing an aqueous solution comprising chlorous acid (HClO₂) that can be used as a microbe disinfectant (chlorous acid aqueous solution), chlorous acid (HClO₂) is produced by adding hydrogen peroxide (H₂O₂) in an amount required to produce chlorous acid by a reducing reaction of chloric acid (HClO₃) obtained by adding sulfuric acid (H₂SO₄) or an aqueous solution thereof to an aqueous solution of sodium chlorate (NaClO₃) so that the aqueous solution of sodium chlorate is in an acidic condition. The basic chemical reaction of this method of manufacturing is represented by the following formula A and formula B.

[Chemical 1]

2NaClO₃+H₂SO₄→2HClO₃+Na₂SO₄ (formula A)

HClO₃+H₂O₂→HClO₂+H₂O+O₂↑ (formula B)

Formula A indicates that chloric acid is obtained by adding sulfuric acid (H₂SO₄) or an aqueous solution thereof in an amount and concentration at which the pH value of a sodium chlorate (NaClO₃) aqueous solution can be maintained within acidity. Next, formula B indicates that chloric acid (HClO₃) is reduced by hydrogen peroxide (H₂O₂) to produce chlorous acid (HClO₂).

[Chemical 2]

HClO₃+H₂O₂→2ClO₂+H₂O+O₂↑(formula C)

2ClO₂+H₂O₂→2HClO₂+O₂↑ (formula D)

2ClO₂+H₂O [symbol] HClO₂+HClO₃ (formula E)

2HClO₂ [symbol] H₂O+Cl₂O₃ (formula F)

At this time, chlorine dioxide gas (ClO₂) is generated (formula C). However, from coexisting with hydrogen peroxide (H₂O₂), chlorous acid (HClO₂) is produced through the reactions in formulae D-F.

Meanwhile, the produced chlorous acid (HClO₂) has a property such that it is decomposed early into chlorine dioxide gas or chlorine gas due to the presence of chloride ion (Cl⁻), hypochlorous acid (HClO) and other reduction substances and a decomposition reaction occurring among a plurality of chlorous acid molecules with one another. Thus, it is necessary to prepare chlorous acid (HClO₂) so that the state of being chlorous acid (HClO₂) can be sustained for an extended period of time in order to be useful as a microbe disinfectant.

In this regard, chlorous acid (HClO₂) can be stably sustained over an extended period of time from creating a transition state to delay a decomposition reaction by adding one compound from inorganic acids, inorganic acid salts, organic acids or organic acid salts, two or more types of compounds therefrom, or a combination thereof to the chlorous acid (HClO₂) or chlorine dioxide gas (ClO₂) obtained by the above-described method or an aqueous solution containing them.

In one embodiment, it is possible to utilize a mixture in which one compound from inorganic acids or inorganic acid salts, specifically carbonate or hydroxy salt, two or more types of compounds therefrom or a combination thereof is added to the chlorous acid (HClO₂) or chlorine dioxide gas (ClO₂) obtained by the above-described method or an aqueous solution containing them.

In another embodiment, it is possible to utilize a mixture in which one compound from inorganic acids, inorganic acid salts, organic acids, or organic acid salts, two or more types of compounds therefrom, or a combination thereof is added to an aqueous solution to which one compound from inorganic acids or inorganic acid salts, specifically carbonate or hydroxy salt, two or more types of compounds therefrom, or a combination thereof is added.

Additionally, in another embodiment, it is possible to utilize a mixture in which one compound from inorganic acids or inorganic acid salts or organic acids or organic acid salts, two or more types of compounds therefrom, or a combination thereof is added to the aqueous solution manufactured by the above-described method.

Carbonic acid, phosphoric acid, boric acid, or sulfuric acid can be used as the above-described inorganic acid. Further, besides carbonate or hydroxy salt, phosphate or borate can be used as the inorganic acid salt. Specifically, sodium carbonate, potassium carbonate, sodium bicarbonate or potassium bicarbonate works well in use as the carbonate; sodium hydroxide, potassium hydroxide, calcium hydroxide, or barium hydroxide works well in use as the hydroxy salt; disodium hydrogen phosphate, sodium dihydrogen phosphate, trisodium phosphate, tripotassium phosphate, dipotassium hydrogen phosphate, or potassium dihydrogen phosphate works well in use as the phosphate; and sodium borate or potassium borate works well in use as the borate. Furthermore, succinic acid, citric acid, malic acid, acetic acid, or lactic acid can be used as the organic acid. Further, sodium succinate, potassium succinate, sodium citrate, potassium citrate, sodium malate, potassium malate, sodium acetate, potassium acetate, sodium lactate, potassium lactate, or calcium lactate is suitable as the organic acid salt.

When an acid and/or a salt thereof is added, a transition state, such as Na⁺+ClO₂⁻<->Na—ClO₂, K⁺+ClO₂⁻<->K—ClO₂, or H⁺+ClO₂⁻<->H—ClO₂ can be temporarily created. This contributes to a delay in the progression of chlorous acid (HClO₂) to chlorine dioxide (ClO₂), which enables the manufacture of an aqueous solution comprising chlorous acid (HClO₂) that sustains chlorous acid (HClO₂) for an extended time and generates a reduced amount of chlorine dioxide (ClO₂).

The following represents the decomposition of chlorite in an acidic aqueous solution.

[Chemical 3]

5ClO₂⁻+→4H⁺→4ClO₂+5Cl⁻+2H₂O (5NaClO₂+4CH₃COOH→4ClO₂+4CH₃COONa+NaCl+2H₂O) (a)

3ClO₂⁻→2ClO₃⁻+Cl⁻(3NaClO₂→2NaClO₃+NaCl)^{Autodecomposition} (b)

ClO₂⁻→Cl⁻+2O (c)

As represented in the formula, the rate of decomposition of a chlorite aqueous solution is greater when pH is lower, i.e., more acidic. That is, the absolute rates of the reactions (a), (b), and (c) in the above-described formula increase. For example, although the ratio accounted for by reaction (a) decreases when pH is lower, the total decomposition rate changes significantly, i.e., to a larger value. Thus, the amount of generated chlorine dioxide (ClO₂) increases with the decrease in pH. Thus, the lower the pH value, sooner the sterilization or bleaching takes effect. However, stimulatory and harmful chlorine dioxide gas (ClO₂) renders an operation more difficult and negatively affects the health of a human being. Further, a reaction of chlorous acid to chlorine dioxide progresses quicker to render chlorous acid unstable. In addition, the time a sterilizing effect can be sustained is very short.

When the above-described inorganic acids, inorganic acid salts, organic acids or organic acid salts are added to an aqueous solution comprising chlorous acid (HClO₂), pH values are adjusted in the range of 3.2-8.5 from the viewpoint of balancing suppression of chlorine dioxide generation and sterilizing effect. For example, with respect to microbe disinfection, an effect against gram-positive microbes Staphylococcus aureus was high on the neutral to alkaline side with pH of 6.5 or higher in a preferred embodiment. Further, in a preferred embodiment, an effect against gram-negative microbes, Enterococcus and Pseudomonas aeruginosa was high on the acidic side, pH of 6.5 of lower. Thus, it was surprisingly revealed that a strong acidity level is not necessarily important in microbe disinfection. It is recognized that both gram-positive microbes and gram-negative microbes can be effectively disinfected near pH of 6.5. Further, pH of the microbe disinfectant of the present invention is preferably, but not limited to, less than 7.0 in terms distinguishing from sodium chlorite. The present invention provides an application as a sterilizing agent which was conventionally not available in terms of providing the optimal application in accordance with the subject to be sterilized.

The present invention was demonstrated as having an effect against drug-resistant microbes such as methicillin-resistant Staphylococcus aureus, multidrug-resistant Pseudomonas aeruginosa, and vancomycin-resistant Enterococcus. The sterilizing agent of the present invention is decomposed after use. Thus, it is not possible to consider in principle that a drug-resistant microbe would arise. In addition, although it is not desired to be constrained by theory, despite the optimal pH values against representative drug-resistant microbes currently occurring being different, the microbe disinfectant was demonstrated to act on each such drug-resistant microbe at about the same level of concentrations. Thus, the microbe disinfectant of the present invention is understood as having a universal effect on drug-resistant microbes. Further, the microbe disinfectant of the present invention was revealed to have an effect on all drug-resistant microbes that were tested near pH of 6.5. Thus, it is possible to provide an all-purpose microbe disinfectant (drug-resistant microbe disinfectant) against drug-resistant microbes by appropriately adjusting pH.

Thus, in one embodiment, the present invention provides a microbe disinfectant comprising a chlorous acid aqueous solution, wherein the disinfectant is made with acidity when applied to gram-negative microbes and with neutrality when applied to gram-positive microbes. Preferably, the acidity used in the present invention is pH of 6.5 or lower and neutrality is pH of 6.5 or higher. The microbe disinfectant of the present invention can be manufactured by using any matter described herein and known information, e.g., information in Patent Literature 1 and the like.

In another aspect, the microbe disinfectant of the present invention is provided as a kit comprising a chlorous acid aqueous solution and a pH adjusting agent, e.g., a drug imparting acidity and/or neutrality. Alternatively, the microbe disinfectant of the present invention is provided at pH of about 6.5. In this case, it is understood that the microbe disinfectant of the present invention is effective on both gram-positive microbes and gram-negative microbes. A pH adjusting agent, e.g., an agent imparting acidity and/or neutrality can be practiced by using any matter described herein and known information.

In one embodiment, microbes targeted by the present invention comprise pathogenic microbes. Thus, the present invention is effective in clinical practice. Microbes on which the present invention is effective include but not limited to E. coli, Staphylococcus aureus, microbes of genus Bacillus, microbes of genus Paenibacillus, Pseudomonas aeruginosa, Enterococcus, Salmonella enterica, Campylobacter, and periodontal disease microbes. Thus, in one embodiment, the present invention also provides a periodontal disease microbes disinfectant comprising a chlorous acid aqueous solution.

In one aspect, the present invention provides a microbe disinfectant comprising a chlorous acid aqueous solution, wherein the disinfectant is made to contact target microbes at a concentration of at least 25 ppm upon contact. It was not possible to predict from conventional results that target microbes can be disinfected at such a low concentration.

In a preferred embodiment, the concentration is at least 50 ppm. The present invention has demonstrated that representative enterohemorrhagic E. coli (O157, O111, O26 and the like) and Salmonella enterica could be disinfected with one minute of contact if chlorous acid is 50 ppm or higher upon contact. Staphylococcus aureus could be disinfected in five minutes with a concentration of 50 ppm at the time of contact. Since the setting of such a concentration at the time of contact can be found from the approximate volume of a target, it is possible to achieve the setting by calculating a suitable amount based on the final volume.

Any reference document cited herein, such as a scientific article, patent and patent application, is incorporated by reference in the present specification in the same manner as the entire contents are specifically described therein.

As described above, the present invention has been explained while presenting preferable embodiments to facilitate understanding. Hereinafter, the present invention is explained based on the Examples. However, the aforementioned explanation and the following Examples are provided solely for exemplification, not for limiting the present invention. Thus, the scope of the present invention is not limited to the Embodiments or Examples that are specifically described herein. The scope of the present invention is limited solely by the scope of the claims.

### EXAMPLES

When necessary, animals used in the following Examples were handled in compliance with the Declaration of Helsinki. For reagents, the specific products described in the Examples were used. However, the reagents can be substituted with an equivalent product from another manufacturer (Sigma, Wako Pure Chemical Industries, Nacalai Tesque, or the like).

(Sample Microbes)

In the present Examples, the following representative microbes were used. The microbes in Example 7 are shown in Example 7.

Periodontal disease microbes: Fusobacterium nucleatum F-1 (selected medium: BHI agar medium)

Methicillin-resistant Staphylococcus aureus: Methicillin-resistant Staphylococcus aureus COL (MRSA; selected medium: BHI agar medium)

Multidrug-resistant Pseudomonas aeruginosa: Multidrug-resistant Pseudomonas aeruginosa TUH (MDRP; selected medium: BHI agar medium)

Vancomycin-resistant Enterococcus: Vancomycin-resistant Enterococcus faecalis BM1447 (VRE; selected medium: BHI agar medium)

(Quantification Method of Chlorous Acid Aqueous Solution)

5 g of the present product is precisely measured. Water is added thereto so that the solution is exactly 100 ml. After 20 ml of the sample solution is accurately measured, put in an iodine flask, and added with 10 ml of sulfuric acid (1→10), 1 g of potassium iodide is added thereto. The flask is immediately sealed and shaken well. A potassium iodide test solution is poured into the top portion of the iodine flask and left standing in the dark for 15 minutes. The plug is then loosened to pour in a potassium iodide test solution and sealed immediately. After sealing and shaking the flask well, freed iodine is titrated with 0.1 mol/L sodium thiosulfate (indicator, starch indicator). The indicator is added after the color of the solution has changed to a light yellow color. A blank test is separately conducted for correction (1 mL of 0.1 mol/L sodium thiosulfate solution=1.711 mg of HClO₂).

### Example 1

### Production of Chlorous Acid Aqueous Solution

The chlorous acid aqueous solution formulation used in the following Example was produced as follows. There are cases herein where an abbreviation “CAAS” is used for a chlorous acid aqueous solution. However, they have the same meaning.

Component Analysis Table for Chlorous Acid Aqueous Solution

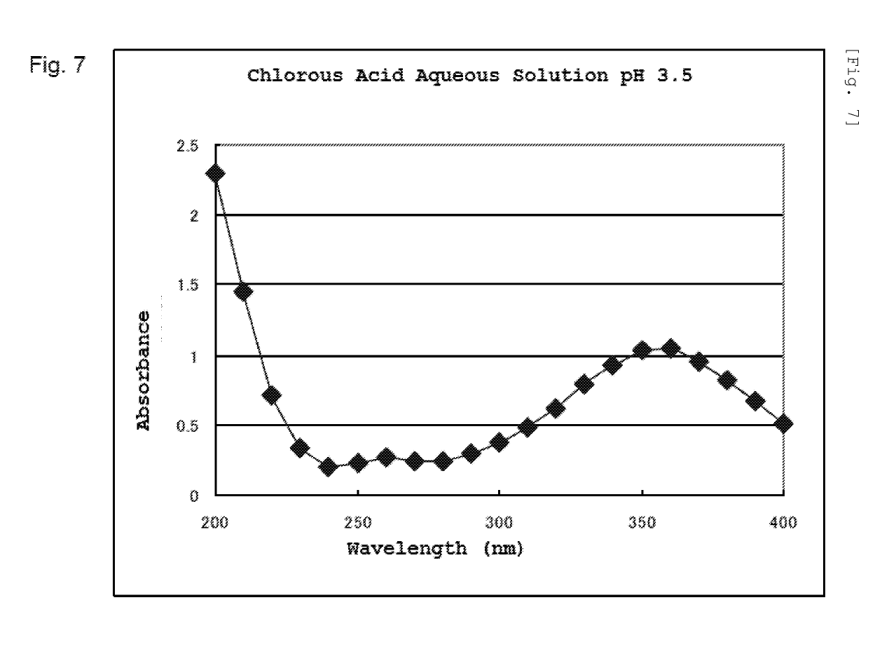

TABLE 2

| CAAS | | Match/Not a |
|---|---|---|
| specification | Specification Value | Match |
| Content | 4-6% | 4.1% |
| Attribute | light yellowish green to yellowish | yellowish red |
| | red | |
| Confirmation Test | When 0.1 ml of potassium | Match |
| (1) | permanganate aqueous solution | |
| | (1→300) is added to 5 ml of an | |
| | aqueous solution of the present | |
| | product (1→20), the solution turns | |
| | reddish purple. When 1 ml of | |
| | sulfuric acid (1→20) is added | |
| | thereto, the solution turns light | |
| | yellow. | |
| Confirmation Test | An aqueous solution of the present | Match |
| (2) | product (1→20) has portions of | The graph for |
| | maximum absorbance at | absorbances |
| | wavelengths 258-262 nm and | and wavelengths |
| | 346-361 nm. | is shown in |
| | | FIG. 7. |
| Confirmation Test | If potassium iodide starch paper is | Match |
| (3) | dipped in the present product, the | |
| | potassium iodide starch paper | |
| | changes to a blue color and then | |
| | the color fades. | |
| Purity Test (1) | 1.0 μg/g or lower for lead | Below detectable |
| | | limit |
| Purity Test (2) | 1.0 μg/g or lower for As₂O₃ | Below detectable |

A chlorous acid aqueous solution formulation was manufactured using this chlorous acid aqueous solution based on the following blend.

TABLE 3

| | | Blended | | Acceptable |
|---|---|---|---|---|
| | Raw material | amount | Concentration | range |
| 1 | Tap water | 258.0 g | | |
| 2 | Dipotassium | 17.0 g | 1.70% | 0.70%-13.90% |
| | hydrogen | | | |
| | phosphate | | | |
| 3 | Potassium | 5.0 g | 0.50% | 0.10%-5.60% |
| | hydroxide | | | |
| 4 | Chlorous acid | 720.0 g | 72.00% | 0.25%-75% |
| | aqueous solution | | | |
| | (pH 3.5) | | | |

| | | |
|---|---|---|
| Total | Chlorous | 1000 g |
| | acid | |
| | 30000 ppm | |

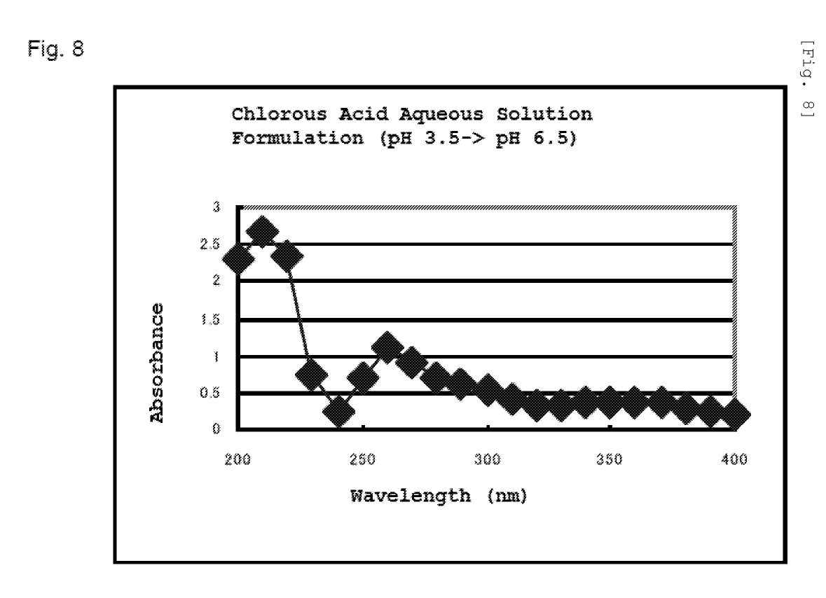

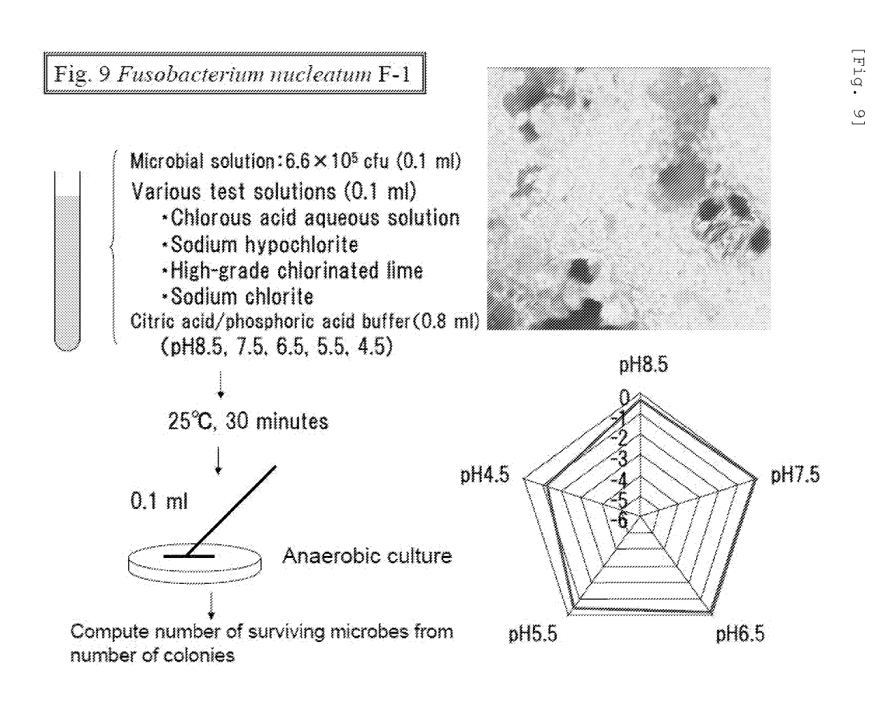

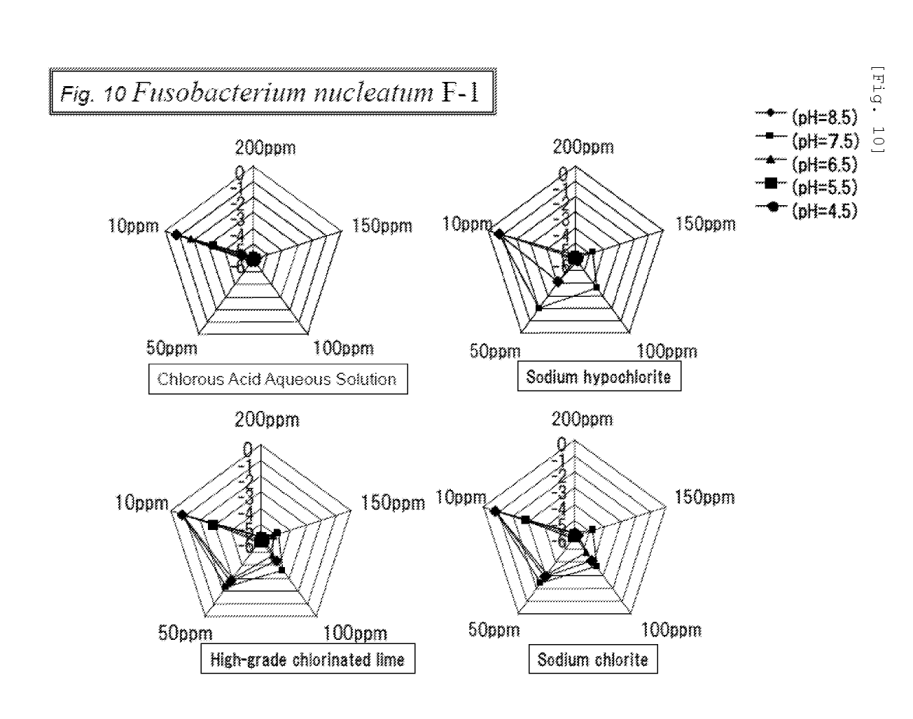

TABLE 4

| | |
|---|---|
| Content | Chlorous acid aqueous solution formulation |
| | manufactured with chlorous acid aqueous |
| CAAS Specification | solution |
| | 3.0% |
| Attribute | Yellow |
| Confirmation Test (1) | Match |
| Confirmation Test (2) | Match (The graph for absorbances and |
| | wavelengths is shown in FIG. 8) |
| Confirmation Test (3) | Match |
| Purity Test (1) | Below detectable limit |
| Purity Test (2) | Below detectable limit |

(Method of Measuring Sterilizing Action (Microbe Disinfecting Action))

Sterilizing (Microbe Disinfecting) Effect of Chlorous Acid Aqueous Solution on Multidrug-Resistant Microbes

The “chlorous acid aqueous solution formulation manufactured with chlorous acid aqueous solution” prepared based on the preparation method of Example 1 was prepared by measuring the concentration of “chlorous acid aqueous solution” based on the above-described quantification method of “chlorous acid aqueous solution” and using each buffer prepared based on the preparation method of buffer so that the available chlorine concentration of “chlorous acid aqueous solution” at the time of contact with tested microbes became 10 ppm, 50 ppm, 100 ppm, 200 ppm, or 500 ppm.

0.1 ml (1-2×10⁹/ml) of test microbial solution (MRSA, MDRP, VRE or the like) was prepared in 0.8 ml of citric acid/phosphoric acid buffer (pH 8.5, 7.5, 6.5, 5.5, or 4.5) and 0.1 ml of test antiseptic agent was prepared. The final concentration was set to 50 ppm, 100 ppm, 200 ppm, 500 ppm or the like. The mixtures were incubated for 30 seconds, one minute, or three minutes at 25° C. The total amount was 0.02 ml.

Next, 0.18 ml of neutralizing solution comprising sodium thiosulfate, polysorbate 80 and lecithin (Difico D/E Neutralizing Broth) was used for neutralization. 0.1 ml was streaked on an LB or BHI agar plate.

(Control Agent)

Sodium chlorite was used as a control agent, which is available from Wako Pure Chemical Industries.

### Example 2

### Effects on Methicillin-Resistant Staphylococcus Aureus COL

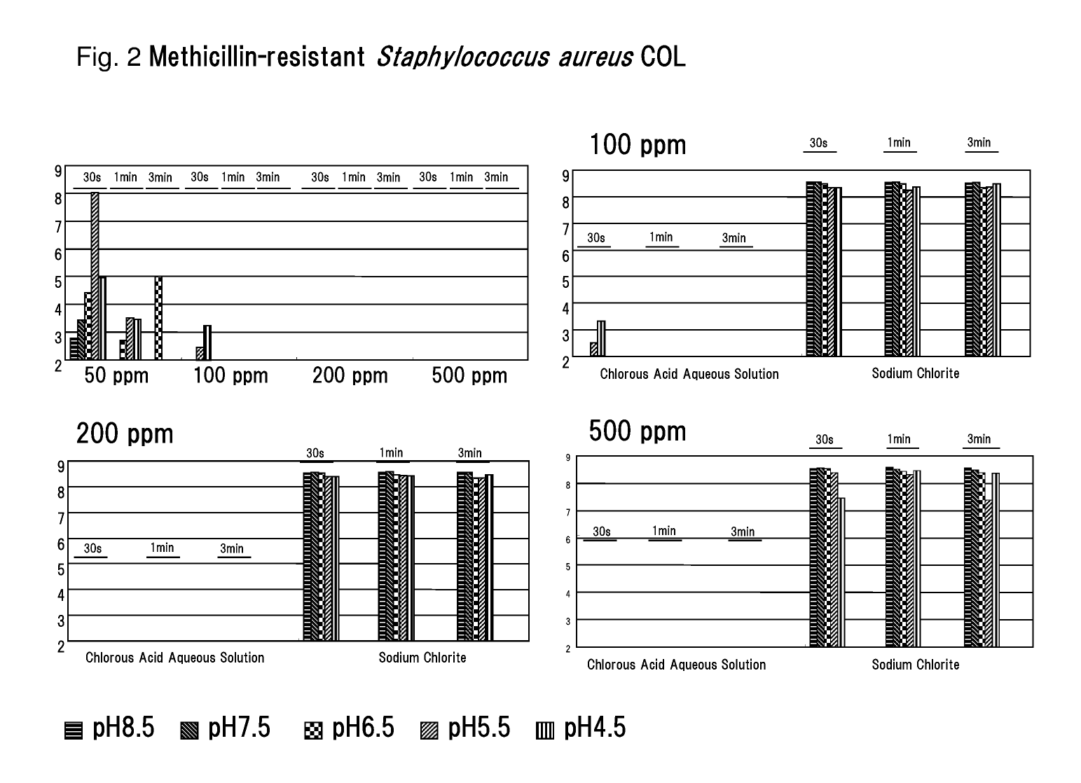

In the present Example, an effect on methicillin-resistant Staphylococcus aureus was examined. The method was in accordance with the above-described (Method of Measuring Sterilizing Action (Microbe Disinfecting Action)). The results are shown in FIG. 2.

As shown, it was demonstrated that MRSA was mostly disinfected at about 100 ppm or higher. It was found that MRSA was completely disinfected in a neutral to alkaline region with a high pH of 6.5 or higher at 100 ppm. From the above, in contrast to prior knowledge, a neutral to alkaline region is understood to be preferable for gram-positive microbes such as MRSA. More specifically, it was found that MRSA was completely disinfected in a neutral region with a high pH of 6.5 to 8.5 at 100 ppm, and considering the distinction from sodium chlorite, pH of 6.5 or higher and less than 7.0. From the above, in contrast to prior knowledge, a pH in the neutral region is understood to be preferable for gram-positive microbes such as MRSA.

### Examples 3

### Effects on Multidrug-Resistant Pseudomonas Aeruginosa TUH

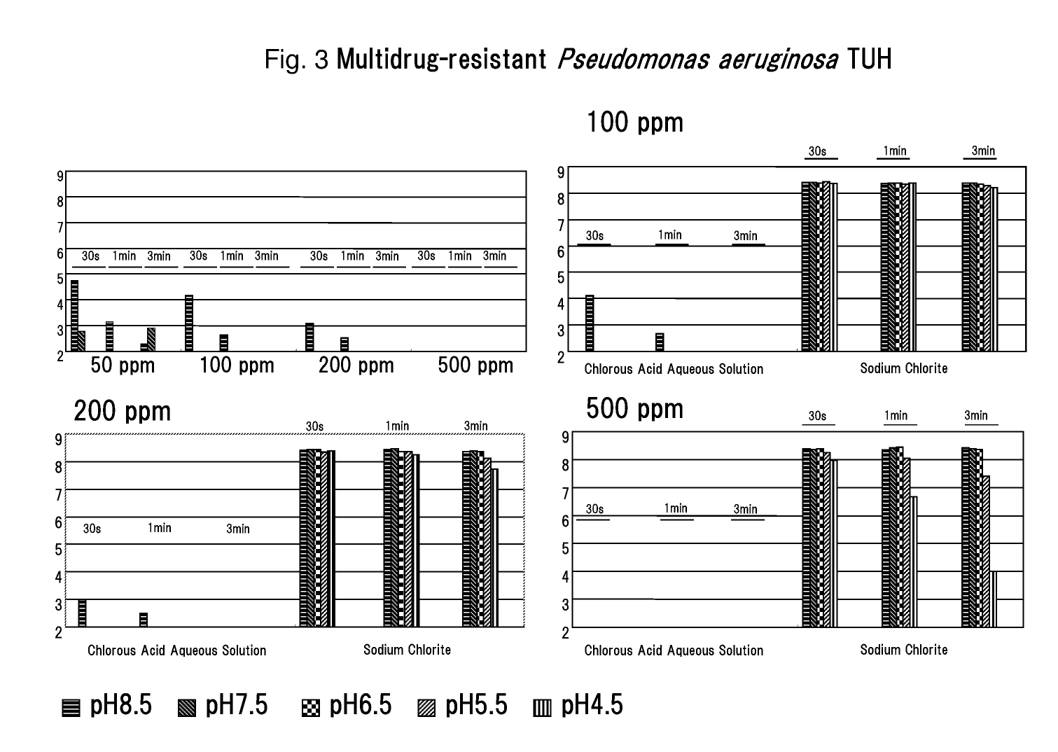

In the present Example, an effect on multidrug-resistant Pseudomonas aeruginosa was examined. The method was in accordance with that described above (Method of Measuring Sterilizing Action (Microbe Disinfecting Action)). The results are shown in FIG. 3.

As shown, it was demonstrated that MDRP was mostly disinfected at about 100 ppm or higher and completely disinfected at 500 ppm. In particular, it was found that MDRP was completely disinfected in an acidic region with a low pH of 6.5 or lower even at 50 ppm. From the above, in contrast to prior knowledge, it was found that an antimicrobial effect had a different preferable pH depending on the microbes.

### Example 4

### Effects on Vancomycin-Resistant Enterococcus faecalis BM1447

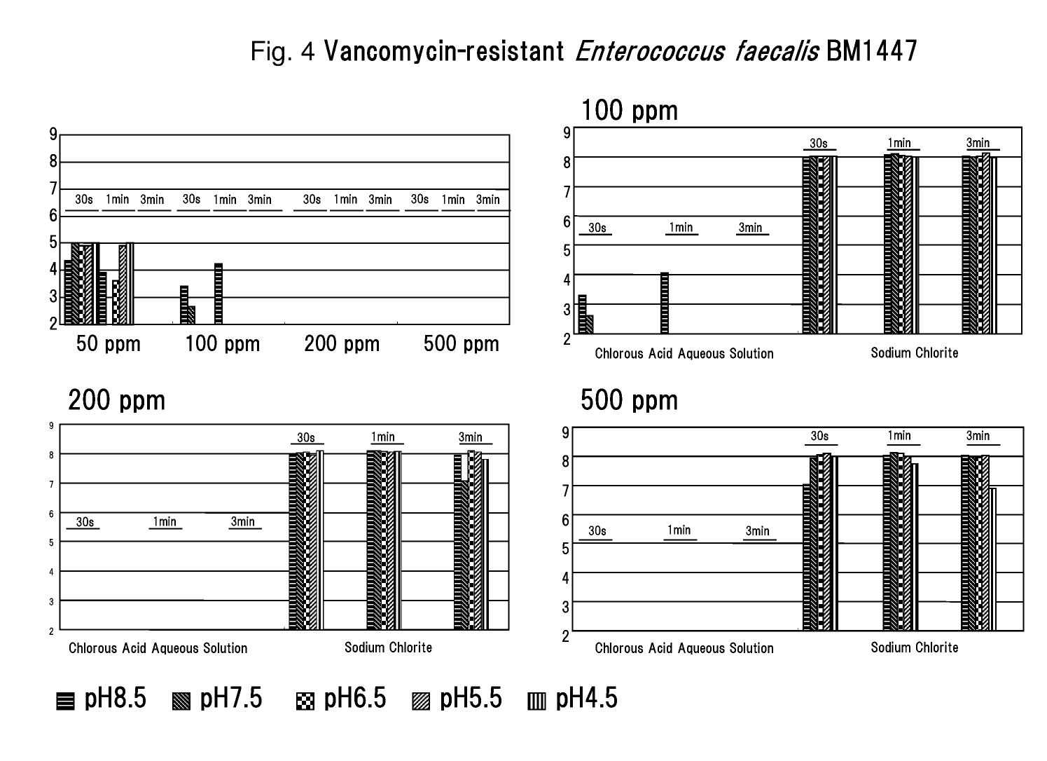

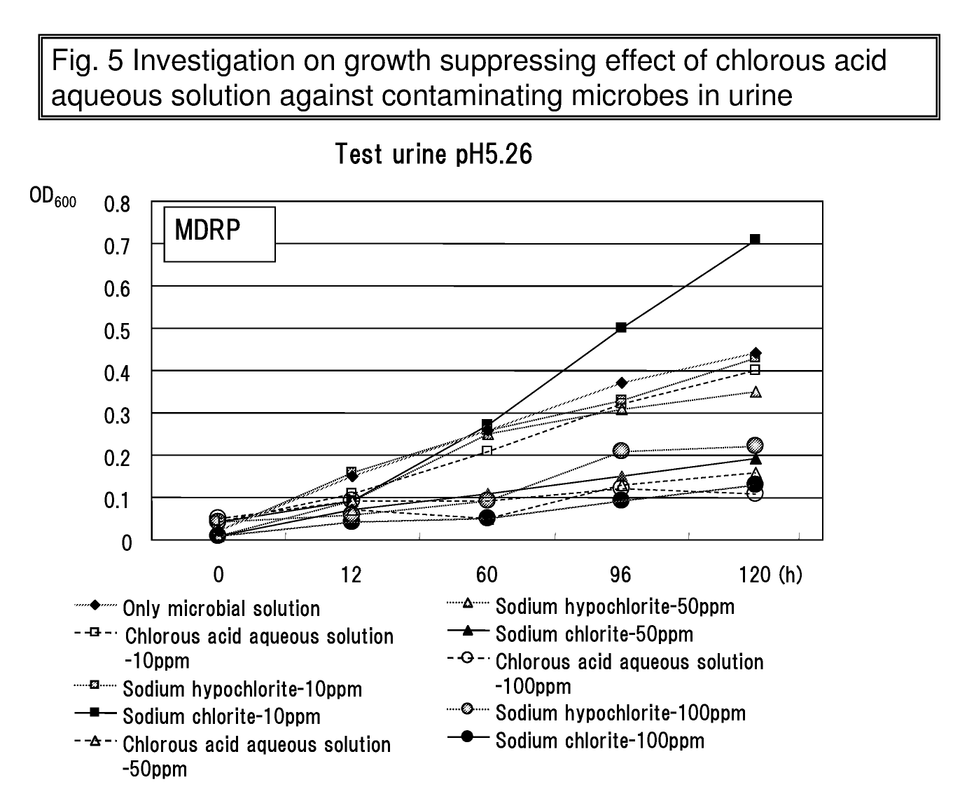

In the present Example, effects on vancomycin-resistant Enterococcus (VRE) were examined. The method was in accordance with that described above (Method of Measuring Sterilizing Action (Microbe Disinfecting Action)). The results are shown in FIG. 4.

As shown, it was demonstrated that VRE was mostly disinfected at about 200 ppm or higher. In particular, it was found that VRE was disinfected in an acidic region with a low pH of 6.5 or lower even at 100 ppm. From the above, in contrast to prior knowledge, it was found that an antimicrobial effect had a different preferable pH depending on the microbes.

(Summary of Sterilizing Effects of Chlorous Acid Aqueous Solution on Multidrug-Resistant Microbes)

A chlorous acid aqueous solution exhibited excellent sterilizing capability against three strains of multidrug-resistant microbes, completely disinfecting more than 99% of tested microbe strains in 30 seconds at a concentration of 100 ppm or higher.

The effect of pH on sterilizing effects of a chlorous acid aqueous solution against multidrug-resistant microbes differs depending on the microbial species. A tendency of enhanced sterilizing capability was recognized on the acidic side with pH of 6.5 of lower against gram-positive microbes (MRSA, VRE) and on the neutral to alkaline side with pH of 6.5 or higher against gram-negative microbes.

### Example 5

### Investigation of Growth Suppressing Effect of Chlorous Acid Aqueous Solution Against Contaminating Microbes in Urine

In the present Example, growth suppressing effects of a chlorous acid aqueous solution against contaminating microbes in urine (MDRP) and MRSA were investigated. The testing method was in accordance with the above-described Examples. The samples prepared as stated above were used.

Tests were conducted twice using similar samples.

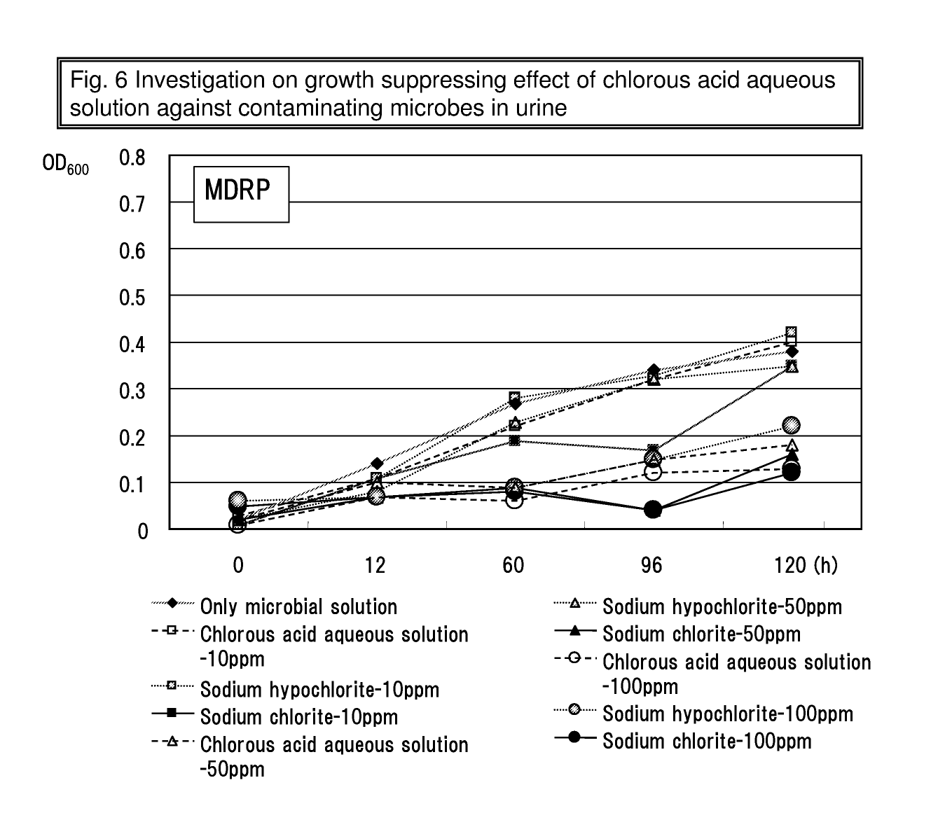

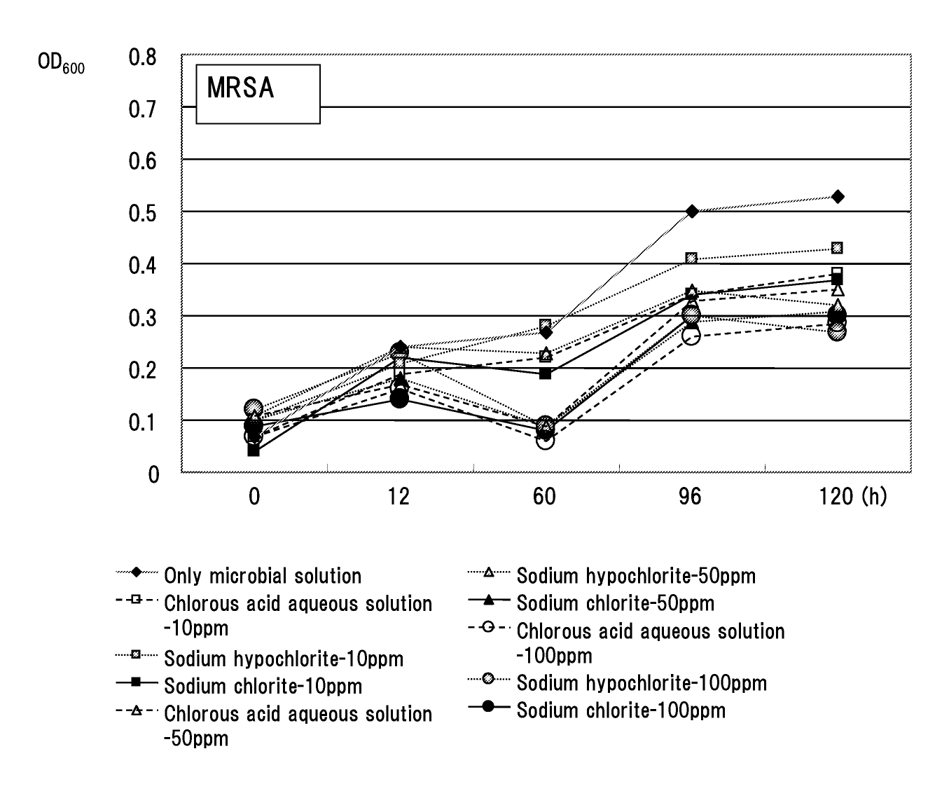

The results are shown in FIGS. 5 and 6. Similar tests were conducted twice and the summary thereof is shown in FIGS. 5 and 6 as test results.

As shown, a growth suppressing effect of MDRP and MRSA similar to those of sodium chlorite and sodium hypochlorite was observed for a chlorous acid aqueous solution.

### Example 6

### Test Results on Periodontal Disease Microbes (Fusobacterium nucleatum F-1))

In the present Example, effects of a chlorous acid aqueous solution on Fusobacteriumnucleatum F-1 as periodontal disease microbes were examined

(Methods)

6.6×10⁵ cfu (0.1 ml) of microbial solution was used. Various test solutions (0.1 ml; chlorous acid aqueous solution; sodium hypochlorite; high-grade chlorinated lime; and sodium chlorite) were used. A citric acid/phosphoric acid buffer (0.8 ml; pH 8.5, 7.5, 6.5, 5.5, and 4.5) was used as buffer. This was an aerobically cultured for 30 minutes at 25° C. The number of surviving microbes was then calculated from the number of colonies.

The results are shown in FIGS. 5 and 6. A chlorous acid aqueous solution exhibited an excellent microbe disinfecting effect against periodontal disease microbes. The effect thereof was equivalent to or greater than that of sodium hypochlorite. The number of surviving microbes was decreased to 10⁻⁵ or less in 30 minutes. Further, there was an effect at 50 ppm against periodontal disease microbes (Fusobacterium nucleatum F-1).

From the above, a chlorous acid aqueous solution is recognized as having an excellent microbe disinfecting effect against periodontal disease microbes.

### Example 7

### Results of Tests for Examining Microbe Disinfecting Effect on Infectious Pathogenic Microbes

In the present Example, tests for examining microbe disinfecting effect on infectious pathogenic microbes were conducted. The methods and results are as shown below.

(Testing Method)

The quantification method of a chlorous acid aqueous solution is as described in the aforementioned (Quantification Method of Chlorous Acid Aqueous Solution).

(Test Microbes that were Used)

1) Enterohemorrhagic Escherichia coli O157: Escherichia coli O157 sakai strain (1996, RIMD0509952)
2) Enterohemorrhagic Escherichia coli O111: Escherichia coli O111 Strain isolated from a patient (2008, RIMD05092028)
3) Enterohemorrhagic Escherichia coli 026: Escherichia coli 026 Strain isolated from mass food poisoning (2000, RIMD05091992)
4) E. coli: Escherichia coli IFO3927
5) Methicillin-resistant Staphylococcus aureus: Methicillin-resistant Staphylococcus aureus COL
6) Staphylococcus aureus: Staphylococcus aureus IFO12732
7) Drug-resistant Pseudomonas aeruginosa: Multidrug-resistant Pseudomonas aeruginosa TUH.
8) Pseudomonas aeruginosa:
9) Vancomycin-resistant Enterococcus: Vancomycin-resistant Enterococcus faecalis BM144710

### 10) Enterococcus:

11) Salmonella enterica: Salmonella Enteritidis IFO3313 *4) E. coli, 6) Staphylococcus aureus, 8) Pseudomonas aeruginosa, and 10) Enterococcus were set for the purpose of reference as indicator microbes upon monitoring at the site.

(Preparation Method of Test Microbes)

1) O157: Enterohemorrhagic Escherichia coli O157: H7 (selected medium: MacConkey medium)
2) O111: Enterohemorrhagic Escherichia coli O111: HNM (selected medium: MacConkey medium)
3) O26: Enterohemorrhagic Escherichia coli O26: H11 (selected medium: MacConkey medium)
4) E. coli: Escherichia coli IFO3927 (selected medium: desoxycholate medium)
The selected medium was streaked. Each tested microbes cultured for 24 hours at 37° C. was suspended in sterile saline to prepare a microbial solution (10⁷ microbes/ml).
5) Salmonella enterica: Salmonella Enteritidis IFO3313 (selected medium: DHL medium)
The selected medium was streaked. Tested microbes cultured for 24 hours at 37° C. were suspended in each sterile saline to prepare a microbial solution (10⁷ microbes/ml).
6) Staphylococcus aureus: Staphylococcus aureus IFO 12732 (selected medium: mannitol salt agar medium with egg yolk) The selected medium was streaked. Each tested microbe cultured for 24 hours at 37° C. was homogeneously suspended in sterile saline to prepare a microbial solution (10⁷ microbes/ml).

(Operational Method)

The “chlorous acid aqueous solution” prepared based on the preparation method was prepared by measuring the concentration of “chlorous acid aqueous solution” based on the above-described quantification method of “chlorous acid aqueous solution” and using each buffer prepared based on the preparation method of buffer so that the available chlorine concentration of “chlorous acid aqueous solution” at the time of contact with tested microbes became 10 ppm, 50 ppm, 100 ppm, 200 ppm, or 500 ppm. 9 ml of each solution was added to a sterilized test tube. These samples were used as sample solutions. 1 ml of microbial solution to be tested was added to the sample solutions and the mixtures were homogeneously mixed. The mixtures were homogeneously mixed again after 1 minute, after 5 minutes, and after 10 minutes, and 1 ml of each mixture was collected. The collection solutions were added to test tubes containing 9 mL of sterilized 0.01 mol/L sodium thiosulfate solution (adjusted with various buffers), homogeneously mixed and neutralized. 0.1 mL of each solution was then collected and spread on one plate of petri dish. About 20 mL of each selected medium was then added. After running pour-plate culture at each temperature and time, the number of surviving microbes was measured.

(Test Results)

Results of Tests for Examining Microbe Disinfecting Effect of “Chlorous Acid Aqueous Solution” against Microbes Causing Infectious Disease and Indicator Microbes of said Microbes Causing Infectious Disease

Table 5. Tests for Examining Microbe Disinfecting Effect of “Chlorous Acid Aqueous Solution” against Enterohemorrhagic Escherichia coli Unit: microbes/ml

TABLE 5

| | | Contact | | | |
|---|---|---|---|---|---|
| | | Con- | | | |
| | | centra- | | | |
| | | tion | | | |
| | Number | Chlorous | | | |
| | of | acid | | | |
| | microbes | concentra- | | | |
| Microbe | as of | tion | Time of contact | | |
| species | contact | (ppm) | 1 min | 5 min | 10 min |
| O157^{1} | 4.1 × 10⁶ | 50 | <100 | <100 | <100 |
| | | 25 | 6.8 × 10³ | 4.9 × 10³ | 4.7 × 10³ |
| O111^{2} | 3.7 × 10⁶ | 50 | <100 | <100 | <100 |
| | | 25 | 2.5 × 10³ | 1.5 × 10³ | 1.2 × 10³ |
| O26^{3} | 2.2 × 10⁶ | 50 | <100 | <100 | <100 |
| | | 25 | 1.1 × 10³ | <100 | <100 |
| Indicator | 2.0 × 10⁶ | 50 | <100 | <100 | <100 |
| microbe^{4} | | 25 | >10⁶ | 3.0 × 10³ | <100 |

*1 Enterohemorrhagic Escherichia coli O157: H7, RIMD0509952, sakai strain isolated in 1996
*2 Enterohemorrhagic Escherichia coli O111: HNM, RIMD05092028, strain isolated from a patient in 2008
*3 Enterohemorrhagic Escherichia coli O26: H11, RIMD05091992, strain isolated from incident of mass food poisoning in 2000
*4 E. coli: Escherichia coli IFO3927

Table 6. Tests for Examining Microbe Disinfecting Effect of “Chlorous Acid Aqueous Solution” against Salmonella enterica

TABLE 6

| | | Contact | | | |
|---|---|---|---|---|---|
| | Number of | concentration | | | |
| | microbes | Chlorous acid | | | |
| Microbe | as of | concentration | Contact Time | | |
| species | contact | (ppm) | 1 min | 5 min | 10 min |
| Salmonella | 1.2 × 10⁷ | 50 | <100 | <100 | <100 |
| enterica | | 25 | >10⁶ | >10⁶ | <100 |

TABLE 6A
Table 6A. Tests for Examining Sterilizing Effect of “AUTOLOC
super” against Staphylococcus aureus

Unit: microbes/ml

| | | Contact | | | |
|---|---|---|---|---|---|
| | Number of | Concentration | | | |
| | microbes | Chlorous acid | | | |
| | as of | concentration | Time of contact | | |
| Microbe species | contact | (ppm) | 1 min | 5 min | 10 min |
| Staphylococcus | 1.0 × 10⁶ | 100 | <100 | <100 | <100 |
| aureus^{6} | | 50 | >10⁶ | <100 | <100 |

*⁶Staphylococcus aureus: Staphylococcus aureus IFO 12732

### Example 8

### Tests for Examining Microbe Disinfecting Effect Against Infectious Pathogenic Microbes Adhering to Chicken Meat

In the Present Example, tests were conducted to examine microbe disinfecting effects against infectious pathogenic microbes. The methods and results are as follows.

(Testing Method)

The quantification method of a chlorous acid aqueous solution is as described in the aforementioned (Quantification Method of Chlorous Acid Aqueous Solution).

(Tested Microbes that were used)

1) Enterohemorrhagic Escherichia coli O157: Escherichia coli O157 sakai strain (1996, RIMD0509952)
2) Campylobacter: Campylobacter jejuni JCM2013

(Preparation Method of Tested Microbes)

1) O157: Enterohemorrhagic Escherichia coli O157: H7 (selected medium: MacConkey medium)

The selected medium was streaked. Each test microbe cultured for 24 hours at 37° C. was suspended in a sterile saline to prepare a microbial solution (10⁹ microbes/ml).

2) Campylobacter: Campylobacter jejuni JCM2013 (selected medium: CCDA plate medium)

The selected medium was streaked. A single colony of tested microbes cultured for 48 hours at 37° C. in microaerophilic conditions was extracted with a platinum loop. The colony was then inoculated in a 50 mL×3 BHI medium, shaken and cultured under aerobic conditions for 48 hours at 37° C. (shake rate: 100 rpm).

(Target Food)

Chicken meat (breast meat): About 2 kg of domestic (location unknown) chicken breast meat that was purchased the day before the tests was used.

(Operational Method)

Each cultured microbial solution was centrifuged (rate of centrifugation: 6000 rpm). The liquid medium of the supernatant was disposed. Then, the microbial solution prepared by diluting with sterilized saline to approximately 10⁷ was put into a manually operated spray in the same amount to make a 10⁶ microbe suspension.

Tests were conducted by the following operational method.

TABLE 7

| | |
|---|---|
| Raw material | Chicken meat (breast meat) |
| Cutting | The chicken meat (breast meat) was cut. |
| Sampling 1 | The number of Campylobacter and Enterohemorrhagic Escherichia coli on the |
| | chicken meat (breast meat) was measured. |
| Microbe | Microbial suspension for spraying (Campylobacter and Enterohemorrhagic Escherichia |
| inoculation | coli) was sprayed on the chicken meat. |
| Sampling 2 | The number of Campylobacter and Enterohemorrhagic Escherichia coli on the |
| | chicken meat (breast meat) was measured. |
| Soaking | Solid liquid ratio Raw material:liquid = 1:10 |
| | Soaking time: 30 minutes |
| | Control *ion exchange water |
| | *chlorous acid aqueous solution, chlorous |
| | acid concentration 400 ppm |
| | *sodium hypochlorite, available chlorine |
| | concentration 400 ppm |
| Sampling 3 | The number of Campylobacter and Enterohemorrhagic Escherichia coli on the |
| | chicken meat (breast meat) was measured. |
| Washing | Sterilized ion exchange water was used for washing. |
| Drain fluid | The liquid was drained in a sterilized draining basket. |
| Sampling 4 | The number of Campylobacter and Enterohemorrhagic Escherichia coli on the |
| | chicken meat (breast meat) was measured. |
| Preservation | Each sterilized chicken meat was stored for 24 hours at 4° C. |
| Sampling 5 | The number of Campylobacter and Enterohemorrhagic Escherichia coli on the |
| | chicken meat (breast meat) was measured. |

*In each sampling section, after 3 samples of chickenmeat (breast meat) were collected, a stomachere was used on about 10 g of the sampled chicken meat. Each suspension was spread on two petridishes to examine the number of residual microbes.

(Test Results)

The results are shown in the following Tables 8 and 9.

Sterilizing Effect on E. coli O-157, Unit: (cfu/mL)

TABLE 8

| | | Chlorous Acid | |
|---|---|---|---|
| | Control | Aqueous Solution | Sodium Hypochlorite |
| Sampling 1 | <100 | <100 | <100 |
| Sampling 2 | 1.8 × 10⁵ | 1.8 × 10⁵ | 1.8 × 10⁵ |
| Sampling 3 | 3.8 × 10⁴ | <100 | 3.3 × 10⁴ |
| Sampling 4 | 4.0 × 10⁴ | <100 | 1.6 × 10⁴ |
| Sampling 5 | 1.3 × 10⁵ | <100 | 5.0 × 10⁴ |

Sterilizing Effect on Campylobacter, Unit: (cfu/mL)

TABLE 9

| | | Chlorous Acid | |
|---|---|---|---|
| | Control | Aqueous Solution | Sodium Hypochlorite |
| Sampling 1 | <100 | <100 | <100 |
| Sampling 2 | 1.4 × 10⁶ | 1.4 × 10⁶ | 1.4 × 10⁶ |
| Sampling 3 | 1.1 × 10⁶ | 6.2 × 10³ | 2.5 × 10⁵ |
| Sampling 4 | 6.2 × 10⁵ | 5.0 × 10³ | 3.2 × 10⁵ |
| Sampling 5 | 8.6 × 10⁵ | 4.3 × 10³ | 4.8 × 10⁵ |

### CONCLUSION/DISCUSSION

As infectious pathogenic microbes, it was demonstrated that Enterohemorrhagic Escherichia coli O157 or Campylobacter can be disinfected.

From the above, it was revealed that a chlorous acid aqueous solution is similarly effective on other gram-negative microbes.

From the results of the Examples, it was revealed that a chlorous acid aqueous solution can be utilized as an effective microbe disinfectant on both gram-positive and gram-negative microbes at pH of 6.5.

### Example 9

### Microbe Disinfecting Effect on Microbes Isolated from Isolator for Raising Microbe-Free Mice

In the present Example, since the isolator for raising microbe-free mice was contaminated by environmental microbes, microbes were isolated from the isolator, and microbe disinfecting effects were examined in vitro using various antiseptics of interest.

<Isolated Microbial Species>

(1) Microbes of the genus Paenibacillus
(2) Microbes of the genus Bacillus

### (3) N. D.

Three species of microbes were isolated to analyze 16SrDNA. As a result, the above-described microbial species were identified. (It was possible to parse out the genus names for Paenibacillus and Bacillus. However, since there were many related species, the name of the species could not be identified. Further, the name of the genus could not be identified for the microbial species of No. (3).

<Agents of Interest>

Control: Sterile ion exchange water
(1) Sodium hypochlorite (Nankai Chemical Co., Ltd.)
(2) “Chlorous acid aqueous solution” formulated in the above-described Examples
(3) Exspor (Ecolab: chlorine dioxide)

<Testing Method>

(1) A single colony of each isolated microbe that was cultured in a BHI agar medium was cultured for two days at 37° C. in 5 mL of BHI medium.
(2) After collecting microbes by centrifuging (3000×g, 4° C., 10 min) the culturedmicrobial solution, the microbes were washed twice with sterile saline (0.85%) to prepare a microbial solution of about 10⁷ CFU/ml (Inoculam value).
(3) Each agent of interest was prepared by diluting with sterile ion exchange water to have a predetermined concentration. 9 mL of each diluent was dispensed into a sterilized test tube.
(4) 1 mL of the microbial solution prepared in (2) was added to the agent-containing test tube. A vortex mixer was used to mix well.
(5) 1 mL was sampled from the test tube of (4) at a predetermined time. The sampling was added to and mixed with 0.05 mol/L sodium thiosulfate solution for neutralization.
(6) 1 mL of the solution after the neutralization treatment was spread on a petri dish. The solution was pour-plate cultured for 24 hours at 37° C. in a BHI agar medium. The number of surviving microbes that had grown was measured.

The operation was performed three times, and microbial disinfection was evaluated by average value±standard deviation (S. D.)

### <Results>

Table 10 Number of Various Microbes that were prepared, Unit: (log₁₀ cfu/mL)

TABLE 10

| | Paenibacillus | Bacillus | N.D. |
|---|---|---|---|
| Inoculum | 6.63 ± 0.13 | 6.42 ± 0.3 | 6.02 ± 0.19 |

Table 11 Results of Sterilizing Effects from Using Various Agents against Microbes, Unit: (log₁₀ cfu/mL)

TABLE 11

| Microbes | Name of Agent | Concentration | 5 min | 10 min | 30 min |
|---|---|---|---|---|---|
| Paenibacillus | Sterile ion exchange water | — | 6.60 ± 0.38 | 6.51 ± 0.29 | 6.52 ± 0.09 |
| | Sodium hypochlorite | 1000 ppm | <2.00 | <2.00 | <2.00 |
| | | 500 ppm | 4.07 ± 0.12 | 3.61 ± 0.09 | 3.61 ± 0.26 |
| | | 200 ppm | 4.80 ± 0.25 | 4.86 ± 0.41 | 3.75 ± 0.53 |
| | Chlorous acid aqueous solution | 1000 ppm | 4.61 ± 0.49 | <2.00 | <2.00 |
| | | 500 ppm | 4.75 ± 0.33 | 4.19 ± 0.24 | <2.00 |
| | | 200 ppm | 5.17 ± 0.58 | 3.16 ± 0.59 | <2.00 |
| | Exspor | — | <2.00 | <2.00 | <2.00 |
| Bacillus | Sterile ion exchange water | — | 6.60 ± 0.38 | 6.80 ± 0.05 | 5.70 ± 0.23 |
| | Sodium hypochlorite | 1000 ppm | 3.55 ± 0.46 | <2.00 | <2.00 |
| | | 500 ppm | 5.55 ± 0.45 | 5.49 ± 0.28 | 4.85 ± 0.32 |
| | | 200 ppm | 5.60 ± 0.22 | 5.53 ± 0.62 | 5.34 ± 0.34 |
| | Chlorous acid | 1000 ppm | <2.00 | <2.00 | <2.00 |
| | aqueous solution | 500 ppm | 3.40 ± 0.28 | 3.14 ± 0.16 | <2.00 |
| | | 200 ppm | 4.74 ± 0.12 | 3.61 ± 0.49 | 3.33 ± 0.41 |
| | Exspor | — | <2.00 | <2.00 | <2.00 |
| N.D. | Sterile ion exchange water | — | 5.84 ± 0.16 | 6.15 ± 0.64 | 5.70 ± 0.23 |
| | Sodium hypochlorite | 1000 ppm | <2.00 | <2.00 | <2.00 |
| | | 500 ppm | <2.00 | <2.00 | <2.00 |
| | | 200 ppm | <2.00 | <2.00 | <2.00 |
| | Chlorous acid | 1000 ppm | <2.00 | <2.00 | <2.00 |
| | aqueous solution | 500 ppm | <2.00 | <2.00 | <2.00 |
| | | 200 ppm | 3.38 ± 0.18 | <2.00 | <2.00 |
| | Exspor | — | <2.00 | <2.00 | <2.00 |

Exspor: Since two agents, a base solution and an activator, are mixed, effects were examined only with an undiluted solution that was made based on the method of use.

Exspor (CLEA Japan, Inc.) used in the above-described Table 10 is a two agent-type sterilizing agent that mixes a BASE (base agent) and an ACTIVATOR (activator) immediately before use. The main ingredient of the BASE (base agent) is sodium chlorite and the main ingredient of the ACTIVATOR (activator) is organic acid. Chlorine dioxide gas that is generated by mixing is used in spraying for gas sterilization. However, since the chlorine dioxide gas cannot be evaluated due to the test format in the present test (in vitro), a mixed solution in which the two agents were mixed was directly used. It is believed that since both chlorous acid and chlorine dioxide, which are a sterilizing component, were present in the mixed solution, a sterilizing effect that is higher than a chlorous acid aqueous solution was obtained as a result.

However, Exspor is inconvenient to use in that preparation at the time of use is imposed. In addition, Exspor is only designed for use by generating chlorine dioxide gas. Thus, an effect of chlorine dioxide gas on a human body is a concern. Meanwhile, a chlorous acid aqueous solution does not impose any inconvenience at the time of preparation because it is only one agent. Further, a chlorous acid aqueous solution generates little chlorine dioxide gas. Thus, a chlorous acid aqueous solution can be used in a safer manner in comparison to Exspor while having almost the same sterilizing effect. In this manner, the chlorous acid aqueous solution of the present invention was demonstrated to enable safe use for exerting the same level of sterilizing effect.

As described above, the present invention is exemplified by the use of its preferred Embodiments and Examples. However, the present invention is not limited thereto. Various embodiments can be practiced within the scope of the structures recited in the claims. It is understood that the scope of the present invention should be interpreted solely based on the claims. Furthermore, it is understood that any patent, any patent application, and any references cited in the present specification should be incorporated by reference in the present specification in the same manner as the contents are specifically described therein.

### INDUSTRIAL APPLICABILITY

The microbe disinfectant comprising a chlorous acid aqueous solution of the present invention can be utilized as a sterilizing agent such as a microbe disinfectant, food additive, antiseptic, quasi-drug, medicine, or the like. Further, it is possible to utilize the microbe disinfectant of the present invention as a more effective sterilizing agent such as a microbe disinfectant, food additive, antiseptic, quasi-drug, medicine, or the like by adjusting the pH.

1.-20. (canceled)

21. A drug-resistant microbe disinfectant comprising a chlorous acid aqueous solution, wherein the drug-resistant microbe disinfectant is formulated for use in inactivating microbes selected from multidrug-resistant Pseudomonas aeruginosa, and vancomycin-resistant Enterococcus, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

22. The drug-resistant microbe disinfectant of claim 21, wherein the drug-resistant microbe disinfectant is present at at least 100 ppm.

23. The drug-resistant microbe disinfectant of claim 21, wherein the drug-resistant microbe disinfectant is present at at least 200 ppm.

24. The drug-resistant microbe disinfectant of claim 21, wherein the drug-resistant microbe disinfectant is present at at least 500 ppm.

25. A drug-resistant microbe disinfectant comprising a chlorous acid aqueous solution, wherein the drug-resistant microbe disinfectant is formulated for use in inactivating methicillin-resistant Staphylococcus aureus and pH is 6.5 or higher, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

26. The drug-resistant microbe disinfectant of claim 21, wherein the drug-resistant microbe disinfectant inactivates microbes selected from multidrug-resistant Pseudomonas aeruginosa and vancomycin-resistant Enterococcus and pH is 6.5 or lower.

27. The drug-resistant microbe disinfectant of claim 21, wherein pH is about 6.5.

28. The drug-resistant microbe disinfectant of claim 21, wherein the drug-resistant microbe disinfectant is a disinfectant for drug-resistant microbes in urine.

29. A microbe disinfectant comprising a chlorous acid aqueous solution, wherein the microbe disinfectant is made with pH of 6.5 or higher when applied to gram-positive microbes, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

30. A combination of microbe disinfectant comprising the microbe disinfectant of claim 29 and a microbe disinfectant comprising a chlorous acid aqueous solution with pH of 6.5 or lower when applied to gram-negative microbes, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

31. The microbe disinfectant of claim 29, wherein the microbe disinfectant is provided as a kit comprising a chlorous acid aqueous solution and an agent imparting acidity and/or neutrality.

32. The combination of microbe disinfectant of claim 30, wherein the microbe disinfectant is provided as a kit comprising a chlorous acid aqueous solution and an agent imparting acidity and/or neutrality.

33. The microbe disinfectant of claim 29, wherein the microbes comprise pathogenic microbes.

34. The combination of microbe disinfectant of claim 30, wherein the microbes comprise pathogenic microbes.

35. The combination of microbe disinfectant of claim 30, wherein the microbes comprises at least one species of microbes selected from the group consisting of E. coli, Staphylococcus aureus, microbes of genus Bacillus, microbes of genus Paenibacillus, Pseudomonas aeruginosa, Enterococcus, Salmonella enterica, Campylobacter, and periodontal disease microbes.

36. A microbe disinfectant for killing periodontal disease microbe comprising a chlorous acid aqueous solution, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

37. The microbe disinfectant of claim 36, wherein pH is about 6.5.

38. A microbe disinfectant comprising a chlorous acid aqueous solution, wherein the disinfectant is made to contact target microbes at a concentration of at least 25 ppm upon contact, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

39. The microbe disinfectant of claim 38, wherein the concentration is at least 50 ppm.

40. A method for disinfecting a drug resistant microbe comprising the step of contacting chlorous acid aqueous solution with a microbe selected from multidrug-resistant Pseudomonas aeruginosa, and vancomycin-resistant Enterococcus, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

41. A method for disinfecting methicillin-resistant Staphylococcus comprising the step of contacting chlorous acid aqueous solution with methicillin-resistant Staphylococcus at pH 6.5 or higher, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

42. A method for disinfecting a microbe comprising the step of contacting chlorous acid aqueous solution with a microbe at pH 6.5 or higher when the microbe is gram positive bacteria, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

43. The method of claim 42 further comprising contacting a chlorous acid aqueous solution with a gram negative microbe at pH 6.5 or lower, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

44. A method for disinfecting a periodontal disease microbe comprising the step of contacting chlorous acid aqueous solution with a periodontal disease microbe, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

45. A method for disinfecting a microbe comprising the step of contacting chlorous acid aqueous solution with a target microbe at a concentration of at least 25 ppm upon contact, wherein the chlorous acid aqueous solution is produced by adding one compound from inorganic acids, inorganic acid salts, organic acids, or organic acid salts, two or more types of compounds therefrom, or a combination thereof to an aqueous solution containing chlorous acid.

46. The method of claim 40, wherein the method is carried out in the presence of organic matter and the disinfecting effect is retained.

47. The method of claim 41, wherein the method is carried out in the presence of organic matter and the disinfecting effect is retained.

48. The method of claim 42, wherein the method is carried out in the presence of organic matter and the disinfecting effect is retained.

49. The method of claim 43, wherein the method is carried out in the presence of organic matter and the disinfecting effect is retained.

50. The method of claim 44, wherein the method is carried out in the presence of organic matter and the disinfecting effect is retained.

51. The method of claim 45, wherein the method is carried out in the presence of organic matter and the disinfecting effect is retained.
